(19) United States
(12) Patent Application Publication
(10) Pub. No.: US 2018/0368996 A1
(43) Pub. Date: Dec. 27, 2018

(54) METHOD FOR PRODUCING A PROSTHESIS SHAFT, AND A PROSTHESIS SHAFT

(71) Applicant: OTTO BOCK HEALTHCARE PRODUCTS GMBH, Wein (AT)

(72) Inventors: Johannis Willem VAN VLIET, Wien (AT); Hans DIETL, Gablitz (AT)

(73) Assignee: OTTO BOCK HEALTHCARE PRODUCTS GMBH, Wien (AT)

(21) Appl. No.: 15/747,093

(22) PCT Filed: Jul. 7, 2016
(86) PCT No.: PCT/EP2016/066169

§ 371 Date: Jan. 23, 2018

(30) Foreign Application Priority Data
Jul. 23, 2015 (DE) 102015112028.3

(51) Int. Cl. A61F 2/80; A61F 2/50

(52) U.S. Cl. CPC A61F 2/80; A61F 2007/0051; A61F 2002/5047; A61F 2/5046

(57) ABSTRACT

A method for producing a prosthesis socket in which a 3D-dataset is produced of an outer contour of a stump on which a prosthesis socket is to be mounted, and a base socket is produced from a first material in a 3D printing method using said 3D dataset, wherein an inner contour of the base socket corresponds to the outer contour of the stump, and at least one stabilising element consisting of a second material is laminated onto the base socket.

The invention relates to a method for producing a prosthesis socket, in which a 3D data record of an outer contour of a stump, to which the prosthesis socket should be applied, is created and a base socket is produced from a first substance in a 3D printing method using the 3D data record, wherein an inner contour of the base socket may correspond to the outer contour of the stump. The invention furthermore relates to a prosthesis socket for receiving a stump of an extremity, having a base socket with an inner contour which corresponds to an outer contour of the stump or which is increased in size by an allowance, wherein the base socket consists of a first substance which is processable in a 3D printing method.

An essential aspect in the production of prosthesis sockets is that, for the purposes of a high comfort of wear, the inner contour of a prosthesis socket corresponds as well as possible or is adapted to the outer contour of an amputation stump. Consequently, each prosthesis socket is ideally produced in an individual manner for the prosthesis wearer, wherein the form of the prosthesis socket depends substantially on the form of the stump and on the expected loads resulting from body weight and activity and the wearing situation.

Various production methods are known for the production of prosthesis sockets. Initially, prosthesis sockets were carved as a wooden socket from a piece of wood. Using more modern manufacturing machines and substances, it was later possible to produce standard sockets made of a metal or polymer. So-called composites were developed which, for example, consist of a combination of a prosthesis socket made of a polymer and a soft liner, which is applied to an amputation stump and which protects the amputation stump from the comparatively hard prosthesis socket.

Producing prosthesis sockets from a laminating resin is known. Here, a curable resin and fiber mats are applied to a plaster model that was made previously by an orthopedic technician and subsequently cured. The socket that arose in this manner is comparatively stiff and unyielding. The socket can be subsequently strengthened using comparatively expensive carbon elements in order to achieve an adequate structural strength. On account of the stiff prosthetic socket, provision should be made of an additional prosthesis liner and a closure system for connecting the prosthesis liner to the prosthesis socket.

A further embodiment of a prosthesis socket provides for a soft internal funnel that is held by a hard carbon frame. Here, the amputation stump, in particular a thigh stump, is embedded in the soft internal funnel in such a way that the prosthesis socket is flexible at its proximal edge and, as a result thereof, the movements of the extremity are only impaired insubstantially. Moreover, the carbon frame has connection means to, for example, a prosthetic knee joint. A plaster model should initially be produced in this type of prosthesis socket as well, making the production complicated and expensive on account of the numerous individual steps as the individual requirements of a prosthesis wearer have to be taken into account.

In order to remedy the known disadvantages, prosthesis sockets have also been produced using the 3D printing method in recent times. In this way, it is possible to allow numerous production steps to run in an automated fashion and hence reduce the outlay and the costs while, at the same time, largely meeting the individual requirements of the prosthesis wearer.

A disadvantage of a prosthesis socket that was produced previously using the 3D printing method is that the structural strength of the processable polymers is unsuitable for a permanent use under very different ambient conditions.

Against this background, it is an object of the present invention to provide a method for producing a prosthesis socket, by means of which it is possible to produce more stable prosthesis sockets that are employable over a wearing time period that is as long as possible.

According to the invention, this object is achieved by a method for producing a prosthesis socket having the features of the main claim and by a prosthesis socket for receiving a stump of an extremity having the features of the coordinate claim. Advantageous embodiments and developments of the invention are disclosed in the dependent claims, the description and the figures.

The method according to the invention for producing a prosthesis socket, in which a 3D data record of an outer contour of a stump, to which a prosthesis socket should be applied, is created and a base socket is produced from a first substance in a 3D printing method using the 3D data record, provides for at least one stabilization element that is made of a second substance to be applied, e.g. laminated, adhesively bonded, welded or fastened in an interlocking manner, onto the base socket. By applying the stabilization element, it is possible to provide the comparatively structurally weak base socket, which was produced in the 3D printing method, with the necessary structural strength in order to allow the prosthesis wearer to wear the prosthesis socket for a wearing duration that is as long as possible. By using different substances for the base socket and the stabilization element, it is possible to take equal account of the individual geometric conditions that are predetermined by the amputation stump and the necessary structural strength. Here, the base socket may be produced from a softer substance and thus further increase the comfort of wear of the prosthesis socket that was already precisely produced in the 3D printing method and matched to the outer contour of the stump. A composite structure which has the necessary structural strength for daily use is formed by applying, laminating, welding, adhesively bonding or otherwise fastening, in an integrally joined, interlocking or frictionally connected manner, at least one stabilization element onto the base socket. As a result of the highly automated production process of the base socket in the 3D printing method and, from a technical point of view, the comparatively simple subsequent application or attachment of the stabilization element, it is possible to produce a marketable and individual prosthesis socket with a high speed and manufacturing quality which were previously not reached in this combination.

A development of the method provides for the 3D data record to be created by a contactless measurement method, in particular an optical measuring using a 3D scanner, specifically with a 3D laser scanner, by evaluating photographs or by way of ultrasound examinations, i.e. overall by a contactless capture of the outer contour of the stump, or by a tactile measurement of the stump. The use of a 3D laser scanner or a digital camera for creating evaluable photographs of the stump has the convenient advantage for the patient that the 3D data record of the outer contour of the stump can be created in a contactless manner. As a result, making complicated molds of the stump, for example by plaster casting, is entirely dispensed with, due to which much time can be saved. The use of a digital camera which is connected to a computer with evaluation software was found to be particularly advantageous. Only two photographs of the stump, for example from a frontal side and lateral side in each case, are necessary for creating the 3D data record. However, it is also possible to make and use more than two photographs, for example photographs in all planes and in the direction of the stump end face at the distal end of the stump. It is also possible to create and further process a circumferential or partly circumferential recording of the stump as a panoramic photograph. A circumferential plane may extend, for example, through a stump center line or perpendicular thereto. The data from the photographs are converted into the 3D data record by way of the evaluation software and may form the basis of the 3D printing method. As a result of only requiring a digital camera and a computer with evaluation software, this provides a particularly cost-effective option of creating the 3D data record. For the 3D data record, it is also possible to use 3D data from a medical examination, for example in a magnetic resonance imaging scanner or from ultrasound examinations. Then, it is possible to resort to already available data and an additional measurement of the stump can be dispensed with completely. As an alternative to the optical, contactless methods, the 3D data record may also be ascertained using a tactile measurement method, for example using a 3D coordinate measuring machine. In this method, a reference point is initially created and, proceeding from this, the further measurement points of the stump are ascertained precisely in relation to one another. The reference point can also be re-ascertained between each further measurement point and a reference to the old reference point may be established. In this way, deviations on account of movements of the stump during the measurement can be better removed from the 3D data record by calculation.

In a development of the invention, the created 3D data record is complemented by entering and/or capturing additional stump data. The created 3D data record of the outer contour corresponds to the subsequently produced inner contour of the base socket. To this end, the inner contour can be manufactured exactly or with necessary tolerances and material allowances. In order to match the socket form and the socket design to the individual requirements and desires of the prosthesis wearer in an ideal manner, it is possible, for example, to determine the bony ML measure and/or the perineum AP measure and feed these into the 3D data record. Moreover, the form of the distal socket end can be designed as a flat or increasingly pointed form, or the prosthesis socket can be constructed with a certain flexion angle and adduction angle. Likewise, a recess for, e.g., a pressure pad in the lateral-posterior region can be provided in the 3D data record and subsequently printed. Furthermore, the form of the subsequent inner contour and/or outer contour of the prosthesis socket can be selected to obtain a desired frictional effect, haptic effect or optical effect. In the 3D printing method, this can be produced, for example, by an offset of individual application layers or a plurality of application layers with respect to one another and/or by way of a plurality of offsets within an application layer such that a surface that is ribbed in the circumferential direction and/or longitudinal direction arises.

In a development of the method, the base socket is produced by polymers or synthetic resins in a 3D printing method according to the principle of polyjet modeling or fused deposition modeling. As a result of a multiplicity of 3D printers already being commercially acquirable for private use from different manufacturers, the costs for 3D printers which operate according to the principle of polyjet modeling or fused deposition modeling are comparatively low. Depending on the size of the 3D printer, it is also possible, in principle, to produce a plurality of individual base sockets in parallel in one print job, which is accompanied by a further time saving and reduction in cost.

In a further embodiment of the method, a receptacle for connection means, for example for an anchor or a connection plate, and/or undercuts or projections for fixing the stabilization element in an improved manner are produced on the base socket during the 3D printing method. To this end, the data for a receptacle are already fed into the 3D data record; i.e., the data record is modified in this respect. The receptacle may include, for example, holes, stops or walls, which are already produced with the base socket in the 3D printing method without further mechanical post-processing of the base socket. Consequently, the time outlay and the costs accompanied therewith for post-processing of the base socket are dispensed with entirely.

Advantageously, the base socket is provided with an inhomogeneous wall thickness over a circumference and/or a longitudinal extent in the 3D printing method. To this end, various allowances or wall thicknesses are assigned to different positions in the created 3D data record of the outer contour such that a non-uniform wall-thickness distribution emerges over the circumference and/or the longitudinal extent of the base socket. In this way, it is possible to form and, in particular, select different wall thicknesses such that more strongly loaded regions are provided with a greater wall thickness than less strongly loaded regions. As a result, the base socket can be configured to be as stable and as light as possible.

Preferably, the outer contour is automatically provided with the inhomogeneous wall thickness. The 3D data record can be automatically adapted to the loads to be expected depending on, for example, the form and spatial orientation of the stump, the weight and/or the shape of the end of the bone of the prosthesis wearer, and the required material thickness can be automatically added at the appropriate points. A further reduction in time and costs can be obtained by the further increased degree of automation. Instead of exactly forming the outer contour of the stump to create the inner contour of the base socket, assigning the outer contour an allowance for the pressure application at selected positions when creating the data record is likewise possible and provided such that a clearance for changes in volume arises at certain positions. Likewise, a reduction in the volume at certain positions may lead to improved contact of the base socket at the stump when, for example, the circumference is reduced in regions.

In an embodiment of the method, the at least one stabilization element is embodied as a frame, a frame part or a layer that surrounds the base socket, and is arranged at, and fastened to, the outer side of the base socket. In this way, the required structural strength of the prosthesis socket can be achieved with as little additional weight as possible and with little material outlay.

Preferably, the at least one stabilization element consists of pre-pregs that are applied to, e.g. laminated on, the base socket and subsequently cured under the influence of pressure and temperature. By way of example, pre-pregs can be textile fibers which are pre-impregnated with a reaction resin. By way of example, they may be present as a unidirectional layer, as a woven fabric or as a laid fabric and may be laminated onto the base socket at the desired position or desired positions in order to increase the rigidity of the base socket. The appropriate positions on the base socket may be ascertained in advance by way of a load simulation, for example with the aid of the finite element method and/or a multi-body simulation, using the 3D data record. In this way, the pre-pregs can be used at an ideal position and save material where possible.

A further embodiment of the method provides for an anchor or a connection plate to be laminated into the base socket. Receptacles or positioning aids for the components to be fastened to the base socket may be embodied on the base socket, preferably on the outer side of the base socket, in order to arrange the components at these positions and connect said components to the base socket, for example by lamination over parts of the stabilization element. As a result of this, the mechanical post-processing of the base socket, of the anchor and/or of the connection plate is advantageously dispensed with almost entirely since only a basic positioning of the components with respect to one another has to be ensured, but not a mechanical locking. As a result, the production costs and the time outlay are reduced further. To the extent that the mechanical loads are not too high, the anchor or the connection plate can also be worked in during the manufacture of the base socket and may be fastened onto or into the latter.

An inner contour of the base socket may correspond to the outer contour of the stump such that the base socket and the stump are embodied in a manner corresponding to one another. This embodiment is advantageous when the base socket contacts the skin surface of the stump directly and it is advantageous, in particular, when using skin-friendly materials for the base socket. Alternatively, it is possible to capture the outer contour of the stump and the immediately arising inner contour may be provided with an allowance such that, in terms of form, the inner contour corresponds to the outer contour of the stump but it would be at a distance from the skin surface in the applied state in order to obtain sufficient space for an interface between the stump and the base socket. The interface may be embodied as a liner, for example made of a silicone material or another polymer; the liner may be coated in order to facilitate an adhesion at the respective surface. It is likewise possible for the interface to have, or be embodied as, a woven fabric or knitted fabric, for example a spacer knitted fabric. The allowance for the inner contour may correspond to the thickness of the interface or it may be slightly smaller in order to ensure a secure seat of the socket on the stump.

The prosthesis socket according to the invention for receiving a stump of an extremity, having a base socket with an inner contour which corresponds to an outer contour of the stump or which is increased in size by an allowance, wherein the base socket consists of a first substance which is processable in a 3D printing method, provides for at least one stabilization element that is applied to the base socket, said stabilization element being made of a second substance which has a higher structural strength than the first substance. As a result, the prosthesis socket according to the invention has a high structural strength and it is matched very precisely to the stump of the prosthesis wearer. In addition to the very economical production, wherein the costs for the 3D printing method may be reduced even further in the coming years, and the fast production, it is possible to take account of the individual requirements of the prosthesis wearer to a great extent and consequently achieve a high standard of quality and comfort. Advantageously, the stabilization element is fastened to the outer side of the base socket, for example laminated, adhesively bonded, welded, fastened in an interlocking manner or fastened in any other manner thereon, such that the inner contour of the base socket is not influenced by the reinforcement. Consequently, the fit remains independent of the form and nature of the stabilization element. The prosthesis socket need not necessarily be manufactured to fit the stump. Where necessary, provision can also be made for a distance between the inner contour and stump, for example for a warming stocking, a liner or a spacer knitted fabric.

In one embodiment of the prosthesis socket, the stabilization element is embodied as a frame, a frame part or a layer that surrounds the base socket. By way of example, the stabilization element can be embodied as a ring or as a plurality of rings that are spaced apart from one another in the longitudinal direction. It is also possible to provide reinforcing ribs between two rings. A layer with a thin wall may be embodied over a large area on the base socket in order to obtain a uniform structure reinforcement. With all embodiments of the stabilization element, it is possible to obtain the required structural strength of the prosthesis socket with a little increase in weight and little material outlay.

In a further embodiment of the prosthesis socket, provision is made for the base socket to have an anchor that is worked in, for example by means of the stabilization element, for example laminated therein, or a worked-in, e.g. laminated-in, connection plate. As a result, the need of mechanical post-processing of the prosthesis socket and/or of the anchor or of the connection place is dispensed with. The connection otherwise obtained by way of e.g. a thread is obtained by simple embedding, laminating-in or other working-in and fastening of the anchor or of the connection plate at or in the base socket. There only has to be basic positioning of the parts with respect to one another beforehand, which is accompanied by an immediate saving of time and costs.

In a preferred embodiment of the prosthesis socket, the base socket has a closed cross section. As a result, the base socket is dimensionally stable and requires less material of the stabilization element for achieving the desired structural strength of the prosthesis socket. As a result of using a polymer or synthetic resin for the base socket, which is cost-effective in comparison with the stabilization element, it is possible to further decrease the material costs.

An exemplary embodiment of the invention will be explained in more detail below on the basis of the figures. In the figures:

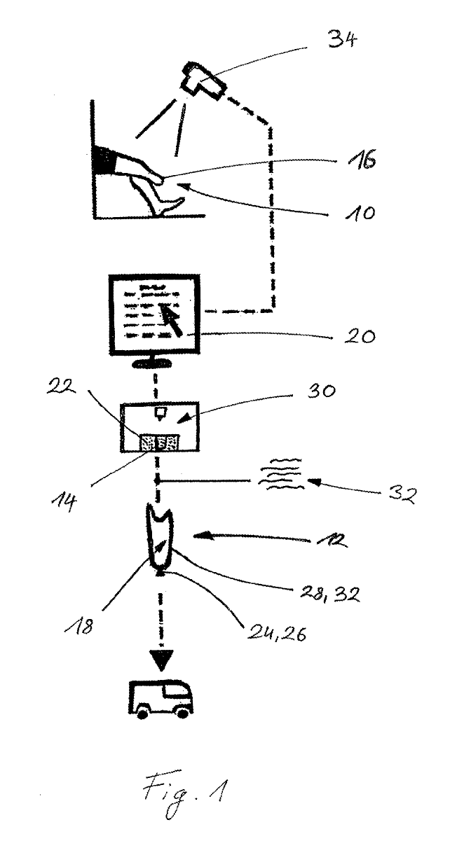

FIG. 1 shows a schematic production process for a prosthesis socket; and

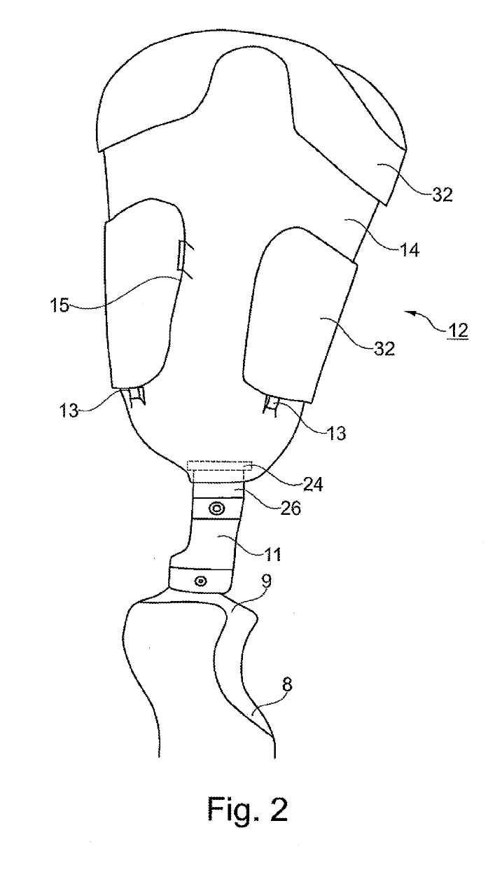

FIG. 2 shows a prosthesis socket with an assembled prosthetic knee joint.

According to the production method for a prosthesis socket that is illustrated in an exemplary manner in FIG. 1, photographs of the outer contour 16 of a stump 10 are initially recorded with the aid of a digital camera 34. Here, it is possible to take two photographs or a plurality of photographs from different directions or, as described above, an at least partly circumferential panoramic image. A 3D data record 20 of the outer contour 16 of the stump 10 is produced from the photographs with the aid of a computer and evaluation software situated thereon. The 3D data record 20, which up until this point only represents the inner contour 18 of the subsequent base socket 14 and of the finished prosthesis socket 12 is subsequently complemented by further data. It was found that, with the aid of appropriate software, e.g. electronic test shaft software, TF or TT design software or CANFIT software by Vorum Research Corporation, a desired socket form and socket design can be produced on the basis of the 3D data record 20 and further desired modifications are quickly implementable. The modified 3D data record 20 is then transmitted to a 3D printer 30. In the 3D printer 30, which operates according to the fused deposition modeling principle in the illustrated embodiment, a polymer 22 or synthetic resin 22 is applied layer-by-layer and fused layer-by-layer to form a base socket 14. In the next method step, the stabilization element 32, which is available in the illustrated embodiment in the form of pre-preg strips or mats, is laminated onto the base socket 14. In the illustrated embodiment, the stabilization element 32 is embodied as a layer 28 that surrounds the base socket 14. Furthermore, a schematically illustrated anchor 24 or a connection plate 26 is laminated into the base socket 14. Subsequently, the assembled base socket 14 is exposed to a pressure and a temperature, which are higher than the ambient pressure and the ambient temperature, respectively, in a chamber that is not illustrated here such that the reaction resin of the pre-pregs cures. After the reaction resin has cured, the prosthesis socket 12 has been completed and it can be supplied to the prosthesis wearer.

FIG. 2 shows the completed prosthesis socket 12 in the assembled state. The base socket 14 has been created on the basis of the 3D data record by means of the 3D printer 30. Here, the inner contour of the base socket 14, which is not illustrated, is matched to the outer contour of the stump, i.e. it exactly corresponds to the outer contour or it was smoothed or provided with an allowance such that an interface or a liner can be arranged between the stump and the inner side of the base socket 14. The outer side of the base socket 14 may have a surface with a substantially smooth wall; in this respect, different wall thicknesses are present in the base socket 14, both over the circumference and over the length of the prosthesis socket 12. Projections 13 and undercuts 15, which serve to position and anchor stabilization elements 32, have been worked into the outer side of the base socket 14. A circumferential first stabilization element 32, for example in the form of a correspondingly formed pre-preg or a plurality of pre-pregs, which are arranged on the semi-stable base socket 14, pre-fixed and then cured under the application of pressure and heat, is arranged and fastened at the upper edge of the base socket 14 in FIG. 2.

A second stabilization element 32, the lower edge of which is seated on the projections 13 and the one side edge of which engages in an undercut 15, is arranged below the first stabilization element 32 which is arranged at the upper edge of the prosthesis socket 12 in a closed circumferential manner. The second stabilization element 32 has a clasp-like contour; i.e. the second stabilization element 32 only partly encompasses the circumference of the base socket 14. Although this brings about an increased stability of the base socket 14 in the region of the second stabilization element 32, it is possible, however, to provide a certain amount of elasticity on account of the open circumferential cross section, even if the material of the stabilization elements 32 has a substantially higher strength against a deformation than the material of the base socket.

Furthermore, FIG. 2 illustrates that the connection plate 26 and the anchor 24 are inserted and fastened or laminated into the base socket 14. In the region of the connection plate 26 and of the anchor 24, provision is made for a material thickening in the base socket 14 at the outer side thereof in order to have improved stability and sufficient space for receiving the components. The material thickening may also be effectuated by way of the stabilization element and/or the coating on the outer side of the base socket 14. In addition to the embedding into the material of the base socket and/or of the stabilization element, the connection plate 26 and the anchor 24 may also be fastened in a conventional mechanical manner, for example by screwing and/or adhesive bonding. Appropriately designed receptacle regions, alignment devices and/or stops may be embodied in the region of the fastening point of the connection plate 26 and of the anchor 24 in order to simplify the positioning of the components. A connection adapter 11 for compensating length is provided at the connection plate 26, said connection adapter in turn being coupled to the prosthetic knee joint 9, which is only indicated, such that the prosthesis socket 12 for receiving a thigh stump is connected to a below-knee part 3 in a pivotable manner.

The base socket 14 may additionally be provided with a coating, which is not illustrated here and which covers the entire outer side of the base socket 14. The coating may be applied in an immersion method or any other application method. Alternatively, the coating may also be applied only to a part of the outer side of the base socket 14; likewise, the coating may be applied, over the whole area thereof or only in part, to the inner side of the base socket 14. By way of the coating, it is possible to adjust the functionality and the surface feel and the surface design of the base socket 14; in particular, it can be adapted to the respective use. The coating and/or the stabilization elements may also be post-treated after the application to the base socket; for example, they may be subjected to heat treatment or they may be irradiated in order to obtain the desired strength properties. Thus, a skin-friendly coating may be applied to the inner side while a dirt-repellent or stabilizing coating may be applied to the outer side of the base socket 14.

### LIST OF REFERENCE SIGNS

8 Below-knee part

9 Prosthetic knee joint

10 Stump

11 Adapter

12 Prosthesis socket

13 Projection

14 Base socket

15 Undercut

16 Outer contour

18 Inner contour

20 3D data record

22 Polymer/synthetic resin

24 Anchor

26 Connection plate

28 Layer

30 3D printer

32 Stabilization element

34 Digital camera

1. A method for producing a prosthesis socket, comprising:
creating a 3D data record of an outer contour of a stump, to which a prosthesis socket should be applied;
producing a base socket from a first substance in a 3D printing method using the 3D data record;
applying at least one stabilization element that is made of a second substance to the base socket.

2. The method as claimed in claim 1, wherein the 3D data record is created by a contactless measurement method or by a tactile measurement of the stump.

3. The method as claimed in claim 1, wherein the 3D data record is complemented by at least one of entering and capturing additional stump data.

4. The method as claimed in claim 1, wherein the 3D printing method is effectuated with at least one of polymers and synthetic resins using polyjet modeling or fused deposition modeling.

5. The method as claimed in claim 1, further comprising producing a receptacle for at least one of a connection member and undercuts or projections for fixing the stabilization element on the base socket during the 3D printing method.

6. The method as claimed in claim 1, wherein the base socket is printed with an inhomogeneous wall thickness over at least one of a circumference of the base socket and a longitudinal extent of the base socket.

7. The method as claimed in claim 6, wherein, in the 3D data record, the outer contour automatically has the inhomogeneous wall thickness applied thereto.

8. The method as claimed in claim 1, wherein the at least one stabilization element is embodied as a frame, a frame part or a layer that surrounds the base socket, and arranged at the base socket.

9. The method as claimed in claim 1, wherein the at least one stabilization element includes pre-pregs that are applied to the base socket and cured under conditions of pressure and temperature.

10. The method as claimed in claim 1, wherein an anchor or a connection plate is worked into the base socket.

11. The method as claimed in claim 1, wherein an inner contour of the base socket corresponds to the outer contour of the stump or the inner contour is increased in size by a thickness of an interface between the inner contour and outer contour.

12. A prosthesis socket for receiving a stump of an extremity, the prosthesis socket comprising:
a base socket with an inner contour which corresponds to an outer contour of the stump or which is increased in size by an allowance, the base socket comprising:
a first substance which is processable in a 3D printing method;
at least one stabilization element applied to the base socket, the stabilization element being made of a second substance which has a higher structural strength than the first substance.

13. The prosthesis socket as claimed in claim 12, wherein the stabilization element is embodied as a frame, a frame part or a layer that surrounds the base socket.

14. The prosthesis socket as claimed in claim 12, wherein the base socket has a worked-in anchor or a worked-in connection plate.

15. The prosthesis socket as claimed in claim 12, wherein the base socket has a closed cross section.

16. A method for producing a prosthesis socket, comprising:
generating an at least partly circumferential panoramic image of an outer contour of a stump;
creating a 3D data record of the stump based at least in part on the image;
producing a base socket from a first substance in a 3D printing method using the 3D data record;
applying at least one stabilization element to the base socket, the at least one stabilization element comprising a second substance.

17. The method as claimed in claim 16, wherein the 3D data record is based at least in part on a contactless measurement method or by a tactile measurement of the stump.

18. The method as claimed in claim 16, wherein the 3D data record is complemented by at least one of entering and capturing additional stump data.

19. The method as claimed in claim 16, wherein the 3D printing method is carried out using at least one of polymers and synthetic resins using polyjet modeling or fused deposition modeling.

20. The method as claimed in claim 16, further comprising producing a receptacle for at least one of a connection member and undercuts or projections for fixing the stabilization element on the base socket during the 3D printing method.
